(19) United States
(12) Patent Application Publication
(10) Pub. No.: US 2015/0081334 A1
(43) Pub. Date: Mar. 19, 2015

(54) METHODS AND SYSTEMS FOR AN ELECTRONIC ASTHMA ACTION PLAN

(71) Applicant: The Charlotte-Mecklenburg Hospital Authority D/B/A Carolinas Healthcare Systems, Charlotte, NC (US)

(72) Inventors: Michael Dulin, Charlotte, NC (US); Lindsay Kuhn, Charlotte, NC (US); Beau Mack, Charlotte, NC (US); Paula Schrum, Charlotte, NC (US); Ron Barus, Charlotte, NC (US); Kelly Reeves, Charlotte, NC (US); Brian Pritchett, Charlotte, NC (US)

(21) Appl. No.: 14/484,553

(22) Filed: Sep. 12, 2014

Related U.S. Application Data
(60) Provisional application No. 61/877,491, filed on Sep. 13, 2013.

(51) Int. Cl. G06F 19/00; A61B 5/00

(52) U.S. Cl. CPC G06F 19/345; A61B 5/4842; G06F 19/322; G06F 19/3431; G06F 19/328; G06F 19/3487; G06F 19/3456
USPC 705/3

(57) ABSTRACT

Methods and systems are provided according to example embodiments of the present invention to provide an electronic asthma action plan/decision support tool as part of an electronic medical record system and to provide a guide to best practices, narrow the complexity of choices, and assist in selecting the appropriate therapy. In some embodiments, such an electronic asthma action plan/decision support tool may provide rapid determination of peak flow expectations; assessment of asthma severity or control; the ability to satisfy asthma appropriate care expectations; provision of guideline-based decision support; electronic prescribing; and generation of an individualized asthma action plan patient handout to support self-management.

### CROSS-REFERENCE TO RELATED APPLICATION

The present application claims the benefit of U.S. Provisional Application No. 61/877,491, entitled “Methods and Systems for an Electronic Asthma Action Plan”, filed on Sep. 13, 2013, the content of which is incorporated by reference herein in its entirety.

### TECHNOLOGICAL FIELD

Example embodiments of the present invention relate generally to providing an electronic asthma action plan decision support tool as part of an electronic medical record system.

### BACKGROUND

Provision of a written asthma action plan (AAP) to patients with asthma is a key component of current guidelines designed to assist patients with self-management of their disease. Although this is considered to be standard of care, provider compliance with this recommendation has generally been limited. In addition, providers often underuse the National Heart, Lung, and Blood Institute (NHLBI) recommended assessments of asthma severity and control, and subsequently, they are unable to use the appropriate prescribing guidelines. One reason underlying poor guideline adherence by providers is likely the complexity of the guidelines. For example, the most recent version is over 400 pages and requires the provider to be able to recall variations dependent upon age, severity or level of control and therapy step for medications.

An asthma action plan generally provides an individualized home management plan of care that is used to guide a patient in how to self-manage their disease at home including what medicine(s) to take, the appropriate dose(s), and frequency. These plans generally also outline what a patient should do in the event of worsening symptoms of an exacerbation and when and/or how to seek help. An asthma action plan may also include trigger identification and avoidance information as well as other important instructions for a patient.

Asthma is a high impact chronic disease that is difficult to manage and associated with disparities in health outcomes, poor medication adherence, and high costs. Asthma prevalence is increasing, and currently affects more than 29.1 million people in the United States, including over 10 million children. There is a great need for innovative approaches to care delivery that facilitate patients' active involvement in their care to improve patient outcomes and reduce costs.

A number of deficiencies and problems associated with more simply and easily providing patients with an asthma action plan are identified herein. Through applied effort, ingenuity, and innovation, exemplary solutions to many of these identified problems are embodied by the present invention, which is described in detail below.

### BRIEF SUMMARY

Methods and systems are provided according to example embodiments of the present invention to provide an electronic asthma action plan/decision support tool as part of an electronic medical record system and provide a guide to best practices, narrow the complexity of choices, and assist in selecting the appropriate therapy. In some embodiments, such an electronic asthma action plan/decision support tool may provide rapid determination of peak flow expectations; assessment of asthma severity or control; the ability to satisfy asthma appropriate care expectations (e.g., clinical quality measures); provision of guideline-based decision support; electronic prescribing; and generation of an individualized asthma action plan patient handout to support self-management.

In one embodiment, a method is provided that at least includes retrieving patient data; receiving an indication of a variable to be used in the patient assessment; determining a patient level of control based at least on the patient data and the indication of the variable; receiving a selection of a therapy step level; determining treatment options for one or more zones based at least on the patient data, the therapy step level, and an applicable guideline; receiving treatment selections for each of the one or more zones; receiving a selection of condition triggers; and generating an asthma action plan based at least in part on the patient data, the level of control, the selected treatment options, the condition triggers, and the applicable guideline.

### BRIEF DESCRIPTION OF THE DRAWINGS

Having thus described certain embodiments of the invention in general terms, reference will now be made to the accompanying drawings, which are not necessarily drawn to scale, and wherein:

FIG. 1 is a diagram of an exemplary system to provide an electronic asthma action plan tool in accordance with an example embodiment of the present invention;

FIG. 2 is a flow chart illustrating operations for an electronic asthma action plan/decision support tool in accordance with an example embodiment of the present invention;

FIGS. 3-13 provide exemplary display interfaces for an electronic action plan tool in accordance with an example embodiment of the present invention; and

FIG. 14 is a block diagram of an apparatus that may be specifically configured in accordance with example embodiments of the present invention.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Embodiments of the present invention provide an electronic asthma action plan/decision support tool as part of an electronic medical record system and provide a guide to best practices, narrow the complexity of choices, and assist in selecting the appropriate therapy. Embodiments of the electronic asthma action plan/decision support tool may help providers overcome the barriers associated with navigating a complex treatment algorithm, drive evidence-based prescribing, and allow for the efficient creation of customized asthma action plans. In some embodiments, such an electronic asthma action plan/decision support tool may auto calculate peak flow zones based on age, height and sex; incorporate medication decision support for identification of medications by zone (e.g., green/yellow/red zones); and provide a patient friendly instruction handout regarding medications and home asthma management.

Embodiments of the electronic asthma action plan/decision support tool may be incorporated within or in communication with an existing EMR and may self-populate with available data such as patient age, height, peak flow and assessment of control, current medications, prior assessments, patient insurance data, etc. Embodiments of the electronic asthma action plan/decision support tool may include built-in decision support which guides providers by self-populating evidence-based guidelines and age appropriate medication choices. Embodiments of the electronic asthma action plan/decision support tool may provide the option to electronically prescribe the selected medications directly to the patient's pharmacy. Embodiments of the electronic asthma action plan/decision support tool may provide for the completed electronic asthma action plan to become a permanent part of the patient's electronic chart and be accessible to other providers within the system.

In some embodiments, the electronic asthma action plan/decision support tool may be configured for use in an outpatient setting. For example, a provider may access or launch the electronic asthma action plan/decision support tool from a content menu such as during patient charting, from a Health Maintenance dashboard display for a patient, or the like, and the provider may complete the electronic asthma action plan during patient consultation and provide the patient with an individualized asthma action plan handout. In some embodiments, the electronic asthma action plan/decision support tool may be configured for use in an acute care setting. For example, the electronic asthma action plan/decision support tool may be accessed as part of an emergency department encounter or inpatient hospitalization standard order set, such as admission or discharge, and produce a Home Management Plan of Care (HMPC) for the patient.

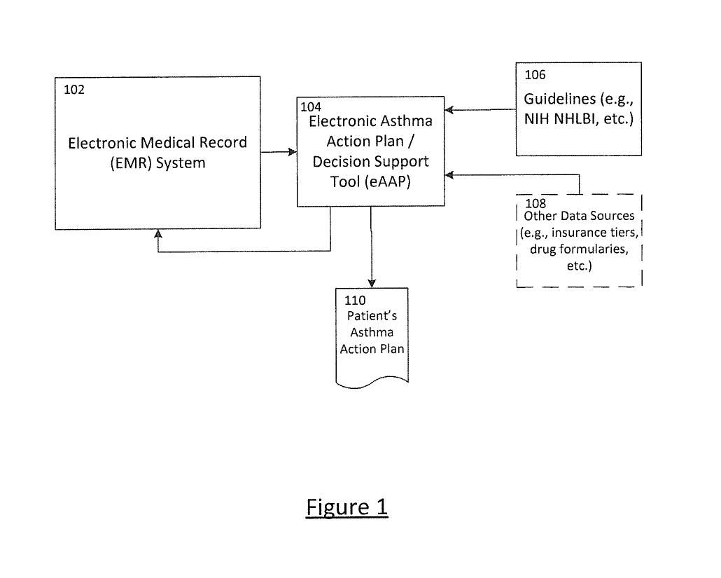

FIG. 1 illustrates exemplary systems to provide an electronic asthma action plan/decision support tool in accordance with an example embodiment of the present invention. A healthcare system may use an electronic medical record (EMR) system, such as EMR system 102, to store data associated with patients. The EMR system may be used by a variety of providers during consultation and treatment of a patient to record patient demographics, medical history, patient's symptoms, past and current medications, treatment, procedures, and the like.

The healthcare system providers may also be provided with an electronic asthma action plan/decision support tool (eAAP), such as eAAP 104, to help providers in navigating complex treatment algorithms and drive evidence-based prescribing and allow for the efficient creation of customized asthma action plans. In some embodiments, the eAAP 104 may provide access to patient demographics for use in assessment; rapid determination of peak flow expectations; assessment of asthma severity or control; the ability to satisfy asthma appropriate care expectations (e.g., clinical quality measures); provisioning of guideline-based decision support; electronic prescribing; and generation of an individualized asthma action plan patient handout to support self-management.

In some embodiments the eAAP may be included as part of an EMR system or embedded within an EMR system, such as EMR system 102 of FIG. 1. In some embodiments, the eAAP may be separate from and in communication with an EMR system. In some embodiments, the eAAP 104 may retrieve patient demographics and, optionally, clinical history from the EMR system 102 for use in the assessment and treatment of a patient. For example, the eAAP 104 may retrieve basic information from the EMR system 102 including a patient's age, height, gender, prior peak flow and assessment of control, and the like for use in assessment and determination of treatment using appropriate guidelines.

In some embodiments, the eAAP 104 may be configured to retrieve past and/or current medical information from the EMR system 102 such as a patient's current medications, immunization history, insurance information, patient pharmacy information, or the like. For example, in some embodiments, the eAAP 104 may also be configured to retrieve the patient's current medication dosages and/or current prescriptions for use in the assessment and treatment decisions. In some embodiments, the eAAP 104 may also be configured to determine what zones and/or treatment steps a patient's existing medications/prescriptions may be associated with when the patient is being assessed using the eAAP for the first time. In some embodiments, the eAAP 104 may also be configured to retrieve a patient's medical insurance information including such information as plan coverage, preferred drugs, payment tiers, non-covered drugs and services, preferred pharmacy, or the like. In some embodiments, the eAAP 104 may be further configured to retrieve patient information regarding one or more of allergies, drug-drug interactions, smoking history or exposure to second-hand smoke, asthma triggers, spirometry results, previous emergency room or inpatient admission dates, oral steroid use, and the like.

In some embodiments, the eAAP 104 may provide decision support which guides providers by self-populating evidence-based guidelines and age appropriate medication choices. To provide such decision support around asthma treatment based on the most recent evidence-based management guidelines, the eAAP 104 may include or access a set of applicable guidelines for the assessment and treatment of patients, such as guidelines source 106. In some embodiments, the eAAP 104 may use guidelines published by a government agency or healthcare organization. For example, in some embodiments, the eAAP 104 may use current national guidelines such as the 2007 National Asthma Education and Prevention Program Expert Panel Report 3: Guidelines for the Diagnosis and Management of Asthma, published by National Institutes of Health National Heart, Lung, and Blood Institute (NIH Publication No. 07-4051, Aug. 28, 2007). In other embodiments, other national or organization guidelines may be used such as the British Guideline on the Management of Asthma (British Thoracic Society, Scottish Intercollegiate Guidelines Network, May 2008, Revised January 2012); the NICE QS25 Quality Standard for Asthma (National Institute for Health and Clinical Excellence, Manchester, England, February 2013), the Canadian Thoracic Society Asthma Guidelines (Canadian Thoracic Society, Asthma Clinical Assembly, www.respiratoryguidelines.ca/guideline/asthma), the Asthma Management Handbook 2006 (National Asthma Council Australia, 2006, www.nationalasthma.org.au), the Global Strategy for Asthma Management and Prevention (Global Initiative for Asthma (GINA), 2012, www.ginasthma.org), or the like. In some embodiments, the eAAP 104 may be configured to use the most recent version of the applicable guidelines.

In some embodiments, the eAAP 104 may also include or access other data sources, such as other data source 108, which may include current drug formularies, insurance medication payment tiers, or the like, for use in developing a patient's asthma action plan.

In some embodiments, the eAAP 104 may produce an individualized asthma action plan patient handout to support self-management, such as patient asthma action plan 110. The individualized asthma action plan patient handout may comprise information on one or more medications the patient should use in each of a plurality of zones along with appropriate dosages and frequencies, how to identify which zone a patient is in based on symptoms and/or peak flow values and the like, what to do if symptoms worsen, and when and how to seek help. The individualized asthma action plan patient handout may further include identification of a patient's triggers and avoidance information, as well as other important instructions for a patient. In some embodiments, the individualized asthma action plan patient handout may also provide authorization information or forms for use by a patient's school. In some embodiments, the eAAP 104 may provide the completed asthma action plan to the EMR system so that the completed asthma action plan becomes a permanent part of a patient's electronic chart and may be accessible to other providers within the healthcare system. In some embodiments, the completed patient asthma action plan may be provided for direct access by a patient, such as through a patient portal or the like. In some embodiments, the completed patient asthma action plan may be provided for direct access to other providers involved in the care of the individual patient, such as through a health information exchange or the like.

While the embodiments of the present invention are described in relation to the treatment of asthma, the invention is not limited to such embodiments. Embodiments of a clinical decision support tool embedded in an EMR or in communication with an EMR may be used for the treatment and management of other chronic illnesses such as chronic obstructive pulmonary disease (COPD), diabetes, heart disease, or the like.

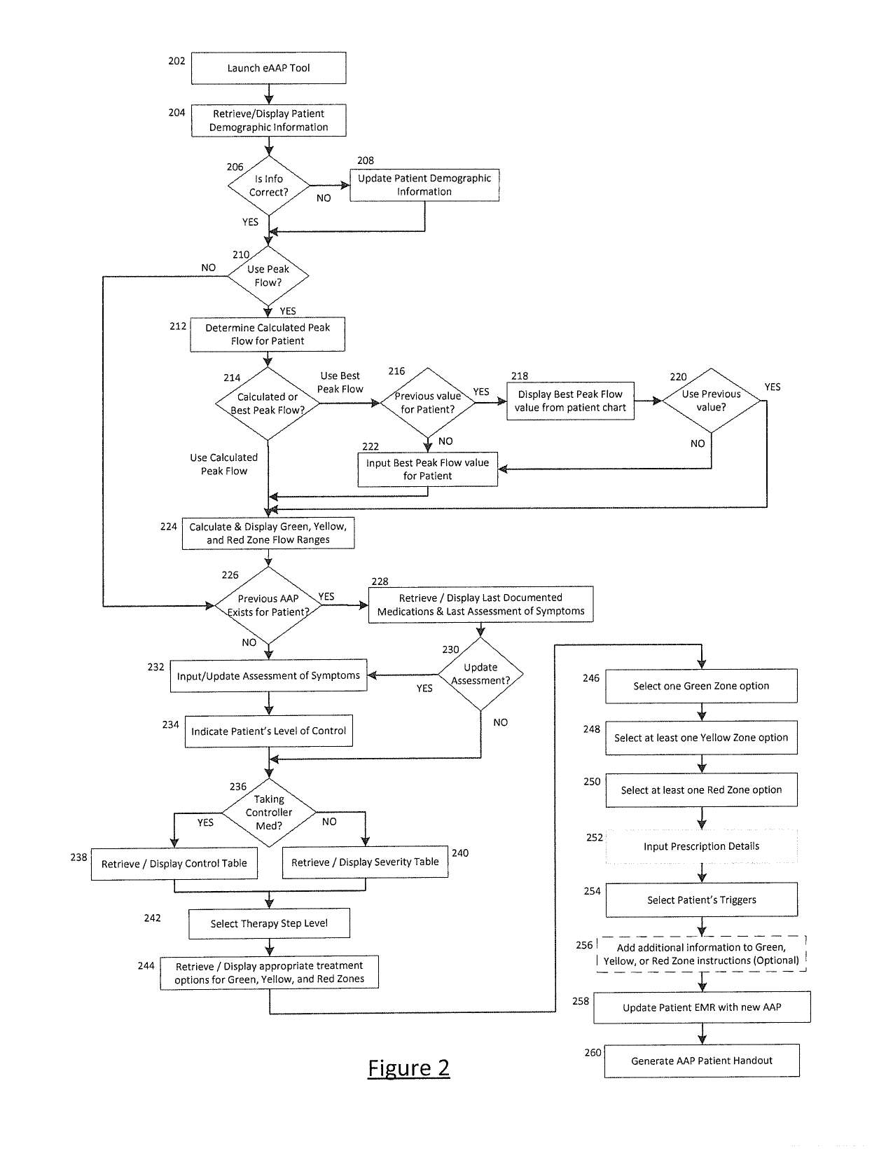

FIG. 2 is a flow chart illustrating operations for an electronic asthma action plan/decision support tool in accordance with an example embodiment of the present invention. In some embodiments, the electronic asthma action plan/decision support tool provides decision support around asthma treatment based on the most recent evidence-based management guidelines and allows providers to assess a patient's control or severity, use a step-wise approach to prescribe medications, complete a detailed Asthma Action Plan patient handout, and satisfy Health Maintenance requirements.

As shown in block 202, operations may begin by receiving instructions from a user (e.g., healthcare provider) to launch the electronic asthma action plan/decision support tool, such as eAAP 104 described in FIG. 1 above. For example, a user may select to launch the electronic asthma action plan/decision support tool from a content menu of an EMR system portal, a Health Maintenance dashboard display for a patient, or the like. In some embodiments, the electronic asthma action plan/decision support tool may be used in an emergency department or inpatient setting and may be accessed as part of a standard order set, such as admission or discharge.

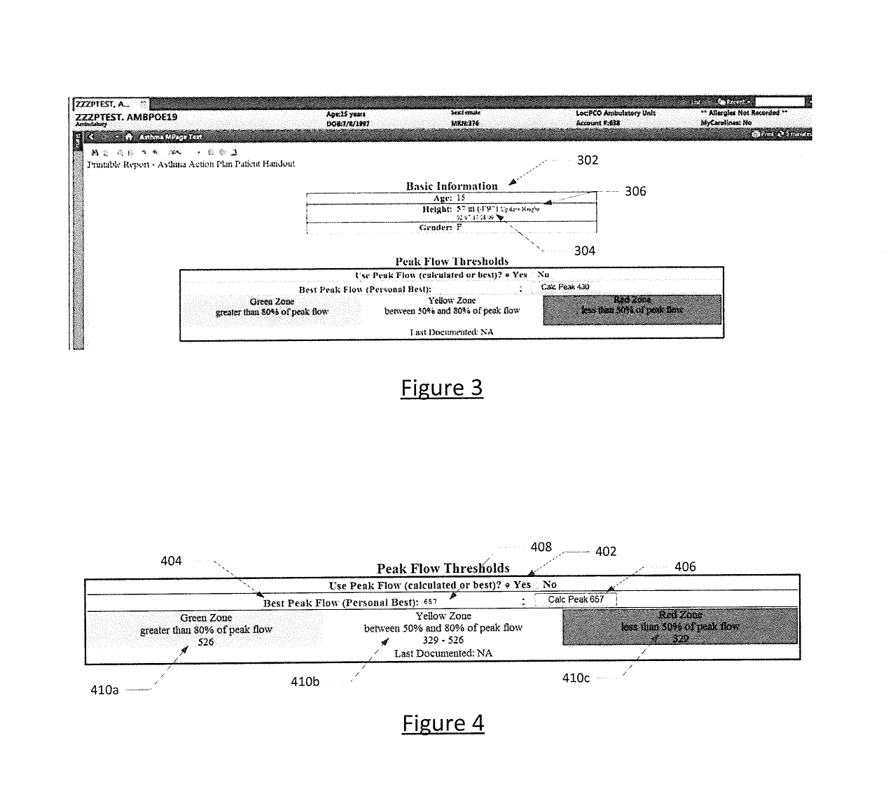

At block 204, the electronic asthma action plan/decision support tool may retrieve and display patient demographic information for use in assessment and the determination of guideline recommendations. In some embodiments, the electronic asthma action plan/decision support tool may retrieve basic information from a patient's EMR, such as from EMR system 102 of FIG. 1, including a patient's age, height, gender, prior peak flow, and assessment of control, and the like. In some embodiments, for example as illustrated in FIG. 3, basic information may be displayed for the patient, including age, height, and gender in Basic Information section 302. The Basic Information section 302 may also display the last date and time 304 that the information was charted for the patient.

In some embodiments, the electronic asthma action plan/decision support tool may also be configured to retrieve information regarding the patient's current medications, including current prescriptions, dosages, etc. for use in the assessment and treatment decisions. In some embodiments, such as when this is the first time a current patient is being assessed, the electronic asthma action plan/decision support tool may be configured to retrieve the patient's current medication information and determine the likely zones and/or treatment steps the current medication is associated with. For example, in some embodiments, the electronic asthma action plan/decision support tool may be configured, such as with custom tables or the like, to recognize a patient's previously prescribed asthma medications the first time the tool is used for the patient and determine the appropriate treatment step the medication should be assigned to so that the provider may easily continue current medications when appropriate.

In some embodiments, the electronic asthma action plan/decision support tool may also retrieve other patient information that may be helpful to the provider's assessment and treatment decisions. For example, in some embodiments, the electronic asthma action plan/decision support tool may retrieve the date of the patient's last flu vaccine from the patient's EMR and display it to the provider. In some embodiments, if the patient does not have a current flu vaccine record or the patient's record needs to be updated, the provider may be provided with a link to an immunization schedule.

At block 206, the electronic asthma action plan/decision support tool may receive an indication from the provider of whether the patient's information is incorrect. If the patient's information is correct, operation continues to block 210. If the provider indicates that the patient's information, such as the patient's height, needs to be updated, such as by clicking a link 306 as illustrated in FIG. 3, the provider may be directed to appropriate interface screens to update the patient's charted information at block 208. Once the patient's information has been updated, operation continues to block 210.

At block 210, the electronic asthma action plan/decision support tool may receive an indication from the provider of whether peak flows should be used in the patient's asthma management. In some embodiments, for example as illustrated in FIG. 4, the provider may indicate whether to use peak flow by making a “Yes” or “No” selection 402. If the provider indicates to not use peak flow, operation may continue to block 226. For example, a provider may indicate to not use peak flows for smaller children and patients unable to perform peak flow measurements. If the provider indicates to use peak flow, operation continues to block 212.

At block 212, the electronic asthma action plan/decision support tool may determine a calculated peak flow for the patient. For example, in some embodiments, the calculated peak flow may be a calculation of the patient's estimated best peak flow based upon his/her basic information and may be determined from an equation or a chart.

At block 214, the electronic asthma action plan/decision support tool may receive an indication from the provider of whether to use a best peak flow or a calculated peak flow for the patient, such as in Best Peak Flow section 404 illustrated in FIG. 4. If the provider indicates to use a calculated peak flow, for example by selecting the “Calc Peak” button 406 illustrated in FIG. 4, the calculated peak flow is used to determine peak flow ranges at operation 224.

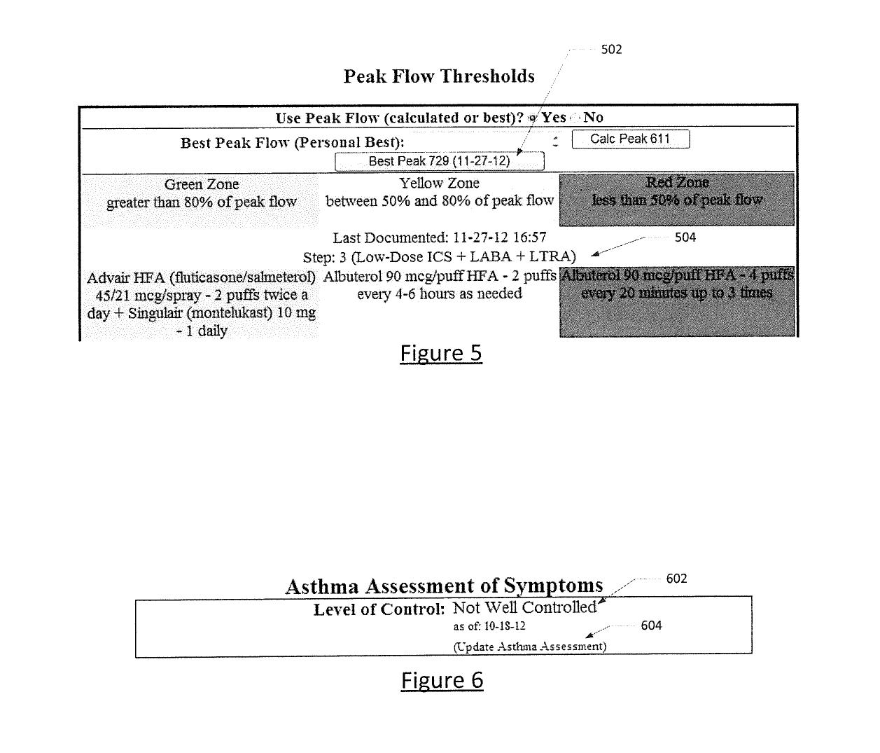

If a best peak flow is to be used, the electronic asthma action plan/decision support tool may determine if a best peak flow has been charted for the patient at block 216. If no best peak flow has been charted for the patient, operation continues to block 222 where the electronic asthma action plan/decision support tool may receive input of a best peak flow to be used. If a best peak flow has been charted for the patient, it may be displayed at block 218. In some embodiments, the patient's last charted best peak flow result along with the date charted may be displayed, for example as illustrated in “Best Peak” button 502 of FIG. 5. In some embodiments, the provider may indicate to use the previous best peak flow measurement by selecting “Best Peak” button 502.

In some embodiments, the provider may indicate that the charted value is not to be used at block 220, such as by manually inputting a different best peak flow value to be used for the patient at block 222. For example, a provider may enter a numerical value (e.g., a value greater than zero) in box 408 illustrated in FIG. 4 to be used as the best peak flow value.

At block 224, the selected calculated peak flow or best peak flow may be used to calculate and display peak flow ranges for each of a green zone, a yellow zone, and a red zone, for example as illustrated in boxes 410a-410c of FIG. 4.

At block 226, the electronic asthma action plan/decision support tool may determine if an asthma action plan has been completed in the past for the patient. If a prior asthma action plan has not been completed, operation may continue to block 232. If a prior asthma action plan has been completed, the last documented medications and last assessment of symptoms may be retrieved and displayed at block 228. For example, in some embodiments, the electronic asthma action plan/decision support tool may cause the last documented medications to be displayed as illustrated in section 504 of FIG. 5 and/or cause the last assessment and the date charted to be displayed as illustrated in section 602 of FIG. 6. In some embodiments, the Asthma Assessment of Symptoms may only be retrieved and displayed if it was charted within a certain time period, such as within the last eighteen months. In some embodiments, the electronic asthma action plan/decision support tool may flag the last Asthma Assessment of Symptoms if it is older than a defined time period, such as older than six months. For example, in some embodiments, the Asthma Assessment of Symptoms may be displayed in a different color or type of text along with an indication that the assessment needs to be updated. Alternatively or additionally, in some embodiments, the electronic asthma action plan/decision support tool may require the assessment to be updated if it is older than the defined time period before operations can continue. At block 230, the electronic asthma action plan/decision support tool may receive an indication to update the asthma assessment of symptoms. In some embodiments, for example, the provider may click a link to indicate that the assessment should be updated, such as link 604 illustrated in FIG. 6. If the assessment is not to be updated, operation may continue to block 236. If the assessment is to be updated operation may continue to block 232.

In some embodiments, even where a prior asthma action plan has not been completed, the electronic asthma action plan/decision support tool may be configured to retrieve the patient's current medication information and make a determination of the likely zone and/or step the medication is associated with.

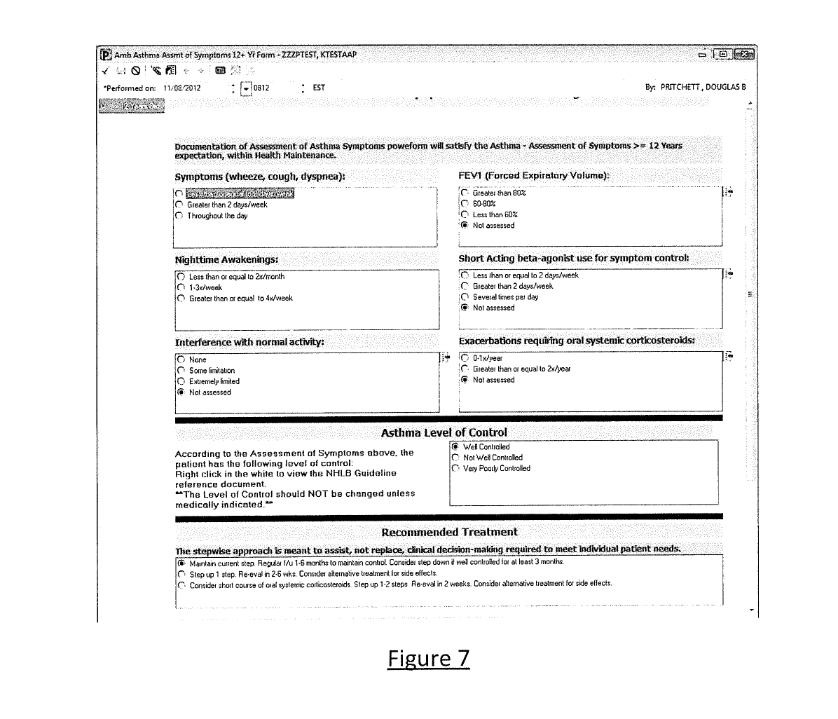

At block 232, the electronic asthma action plan/decision support tool may display a symptoms assessment form, for example such as illustrated in FIG. 7. The electronic asthma action plan/decision support tool may receive the provider's answers to the assessment questions for use in determining the patient's level of control based on guidelines at block 234.

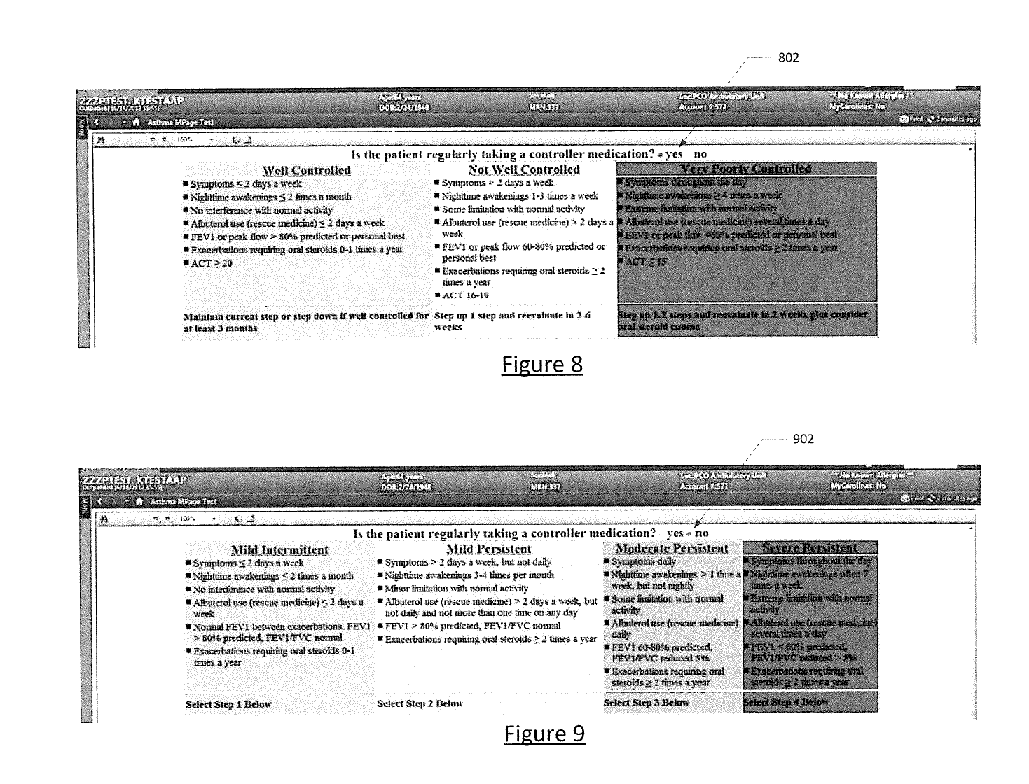

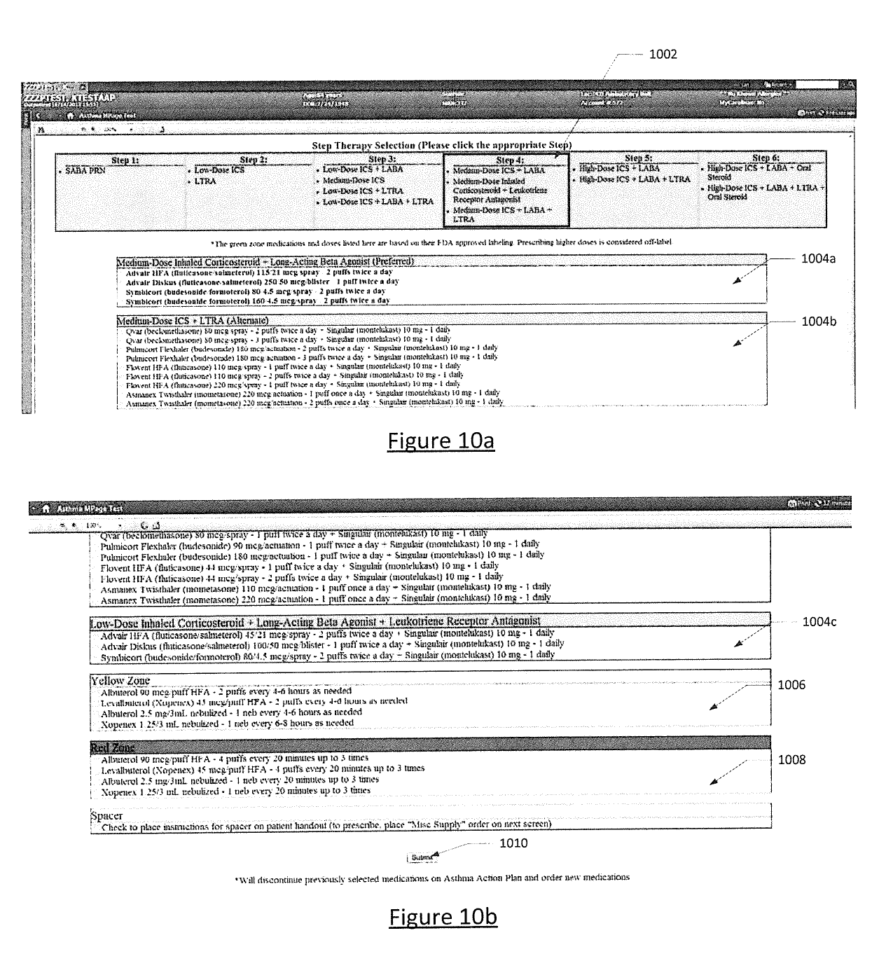

At block 236, the electronic asthma action plan/decision support tool may receive input from the provider of whether the patient is regularly using a controller medication (a medication taken daily to prevent asthma symptoms including wheezing, coughing, chest tightness, and shortness of breath, generally in an inhaler form). If the patient is regularly using a controller medication, such as indicated by selection 802 of FIG. 8, the electronic asthma action plan/decision support tool may display an age specific Control table at block 238, such as illustrated in FIG. 8. If the patient is not regularly using a controller medication, such as indicated by selection 902 of FIG. 9, the electronic asthma action plan/decision support tool may display an age specific Severity table at block 240, such as illustrated in FIG. 9. The provider may then use the Control or Severity table to determine the patient's level of Control (e.g., well controlled, not well controlled, or very poorly controlled) or level of Severity (e.g., mild intermittent, mild persistent, moderate, persistent, or severe persistent) to guide the treatment selection. Generally, Severity may be determined by assessing asthma symptoms, nighttime awakenings, short-acting beta-agonist use for symptom control, interference with normal activity, lung function, and exacerbations requiring oral systemic corticosteroids. Control may be similarly assessed with the additions of FEV1 (forced expiratory volume in 1 second) or peak flow and validated questionnaires (e.g., Asthma Therapy Assessment Questionnaire (ATAQ), Asthma Control Questionnaire (ACQ), or Asthma Control Test (ACT)).

At block 242, the electronic asthma action plan/decision support tool may receive a selection of the appropriate therapy step selected by the provider. For example, in some embodiments, the electronic asthma action plan/decision support tool may display a table of the steps of therapy based on guidelines for selection by the provider, as illustrated in FIG. 10. The provider may select the appropriate step, such as by clicking on the selected step, such as illustrated in selection 1002 of FIG. 10a.

At block 244, the electronic asthma action plan/decision support tool may retrieve and display the appropriate treatments for a plurality of zones, for example, each of a green zone, a yellow zone, and a red zone, based on the guidelines used in the electronic asthma action plan/decision support tool. In some embodiments, the electronic asthma action plan/decision support tool may be configured to determine and display the age and dose specific, evidence-based treatment options for each of the green zone, the yellow zone, and the red zone. For example, in some embodiments, the electronic asthma action plan/decision support tool may be configured to analyze the patient's basic information, the assessment of control/severity, and the selected therapy step to determine the appropriate treatment options. For example, guidelines may provide a stepwise approach to treatment which outlines preferred and alternative regimens to achieve control for various patient demographic groups, with various asthma controller medications assigned to specific “step” categories based on FDA and/or usual dosages. In some embodiments, preferred and alternate medications may be displayed differently within each of the zones. For example, preferred medications may be in a different text, such as boldface, and may appear higher in the list than alternative medications. An example of the zones is illustrated in FIGS. 10a and 10b, with green zone medications 1004a-c, yellow zone medications 1006, and red zone medications 1008. In some embodiments, a clinical decision support element may be bypassed enabling the provider to prescribe off-step or off-label medication options if the provider feels it is in the best interest of the patient.

In some embodiments, the electronic asthma action plan/decision support tool may also be configured to retrieve and display the formulary status of the medications in each zone, and if the medication is on formulary, retrieve and display the patient's insurance payment tiers. For example, in some embodiments, the formulary status and payment tier may be displayed along with the medication in each zone or a link may be displayed with the medication to access the information, such as by displaying in a pop-up window or the like.

At block 246, the electronic asthma action plan/decision support tool receives a selection of one treatment option from the green zone. At block 248, the electronic asthma action plan/decision support tool may receive a selection of one or more treatment options from the yellow zone. At block 250, the electronic asthma action plan/decision support tool may receive a selection of one or more treatment options from the red zone. The electronic asthma action plan/decision support tool may then receive an indication that selections are complete, such as by the selection of submit button 1010 illustrated in FIG. 10b.

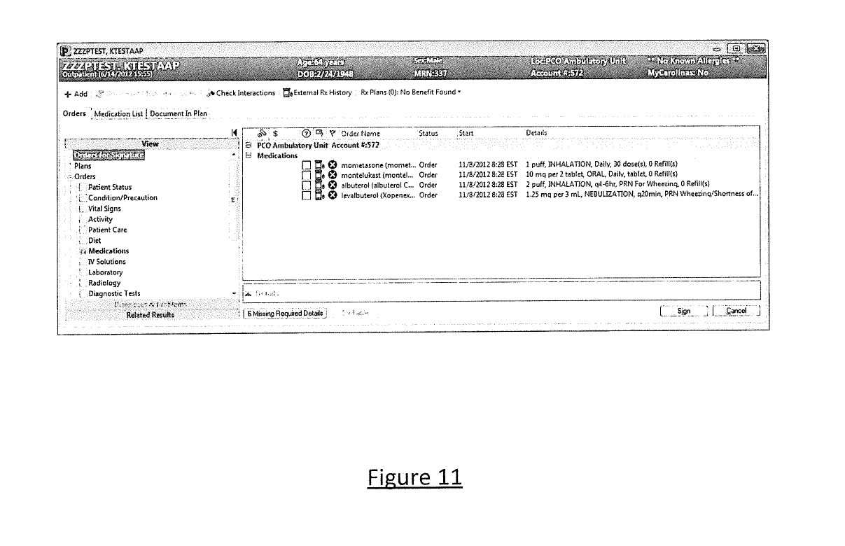

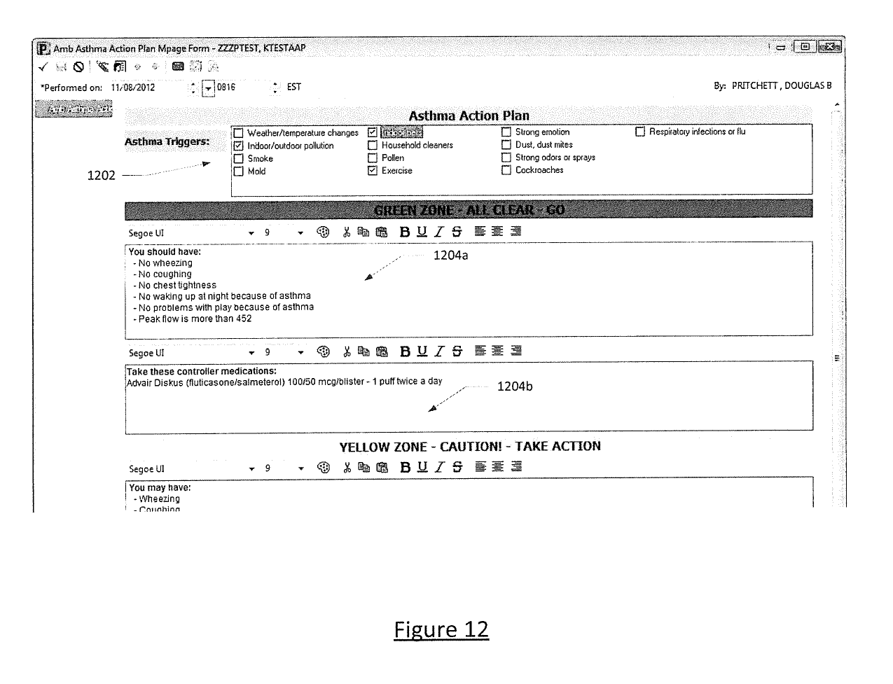

At block 252, the electronic asthma action plan/decision support tool may generate and display a medication ordering screen, such as illustrated in FIG. 11. The electronic asthma action plan/decision support tool may receive input from the provider of any necessary prescription details to complete the medication ordering process. In some embodiments, the electronic asthma action plan/decision support tool may be configured provide for electronic ordering of the patient's prescriptions to the patient's specified pharmacy as well as modification to the routing of the patient's prescriptions, such as to an alternate pharmacy, to generate a printed prescription, to indicate the medications have been called in to the patient's pharmacy, or the like. In some embodiments, the completion of the medication ordering process may also discontinue any previously prescribed medications for the patient.

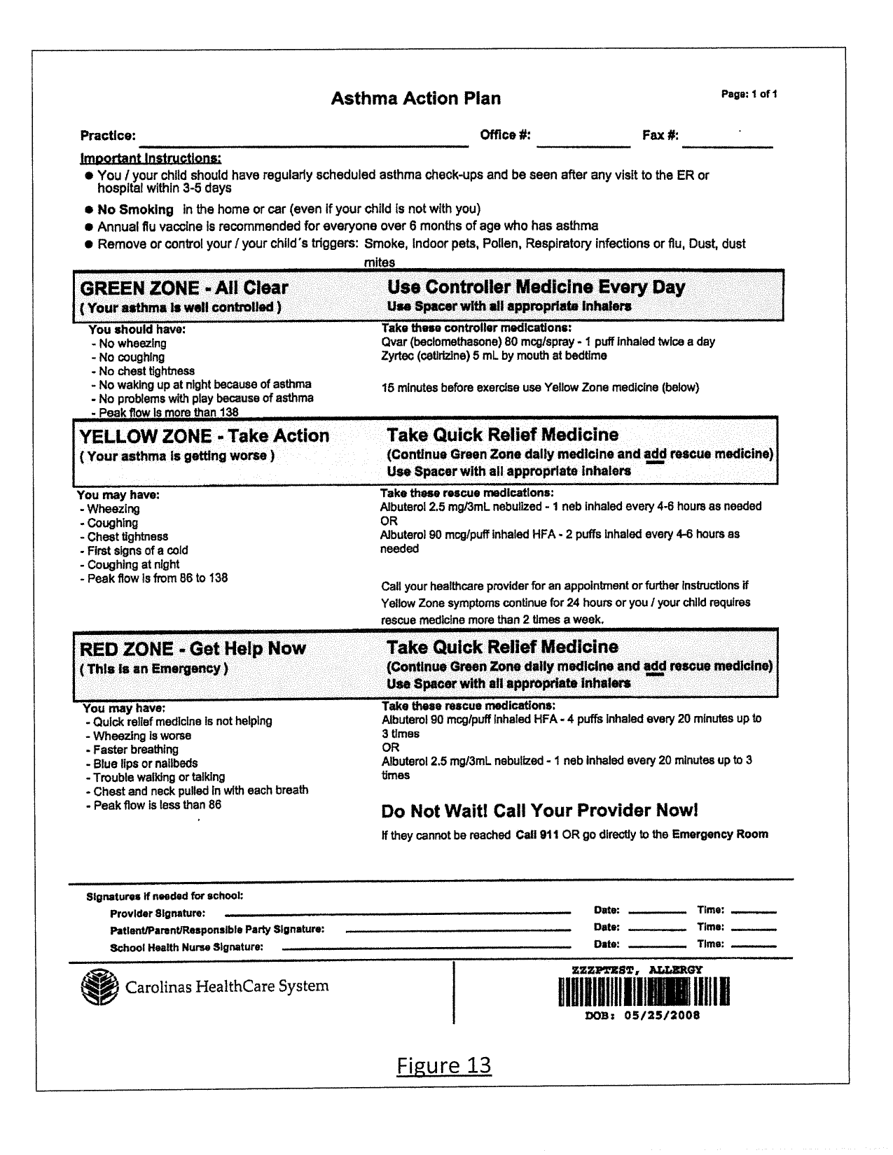

At block 254, the electronic asthma action plan/decision support tool may generate and display a form for inputting a patient's triggers, such as section 1202 illustrated in FIG. 12. The electronic asthma action plan/decision support tool may also display patient information for each of the plurality of zones, including symptoms, medications, instructions, and the like. In some embodiments, at block 256, the electronic asthma action plan/decision support tool may allow for a provider to edit and/or add additional text to each of the green, yellow, and red zones for the patient, such as sections 1204a-b illustrated in FIG. 12.

At block 258, the electronic asthma action plan/decision support tool may receive an indication from the provider that the electronic asthma action plan is complete and the patient's EMR may be updated with the new electronic asthma action plan. In some embodiments, the electronic asthma action plan/decision support tool may be configured to verify that all the required sections of the electronic asthma action plan have been completed, and if the plan is incomplete, indicate what sections must be completed. For example, in some embodiments, one or more messages may be provided indicating incomplete sections or the incomplete sections may be highlighted in some manner. In some embodiments, the electronic asthma action plan/decision support tool may be configured such that the operations cannot proceed until the incomplete sections are completed.

At block 260, the electronic asthma action plan/decision support tool may generate the asthma action plan patient handout, such as illustrated in FIG. 13. In some embodiments, the asthma action plan patient handout may include portions to indicate authorization for medication use at the patient's school. In some embodiments, the electronic asthma action plan/decision support tool may be configured to generate the asthma action plan patient handout in a language specified for the patient (e.g., Spanish). In some embodiments, the electronic asthma action plan/decision support tool may be configured to generate the asthma action plan patient handout in a simple black and white print format or in a full color format, such as to highlight the different zones or specific instructions or information. In some embodiments, the electronic asthma action plan/decision support tool may be configured to include a school district-specific form along with the asthma action plan patient handout.

Once the asthma action plan patient handout has been generated and the patient's EMR has been updated with the new electronic asthma action plan, the asthma action plan patient handout may be retrieved by other appropriate staff within the healthcare system, such as for providing additional copies to a patient, etc. In some embodiments, the asthma action plan patient handout may be directly accessible to the patient, such as through a patient portal or the like. In some embodiments, the asthma action plan patient handout may be directly accessible to other providers involved in the care of the patient, such as through a health information exchange or the like.

In some embodiments, the electronic asthma action plan/decision support tool may be further configured to provide data to monitor quality of care and patient engagement, for example by providing data to monitoring dashboard applications or the like. For example, in some embodiments, the electronic asthma action plan/decision support tool may be configured to provide data to be incorporated into other asthma-related metrics for quality assurance (or other appropriate metrics), such as health maintenance, core measures, meaningful use/appropriate care requirements, and the like.

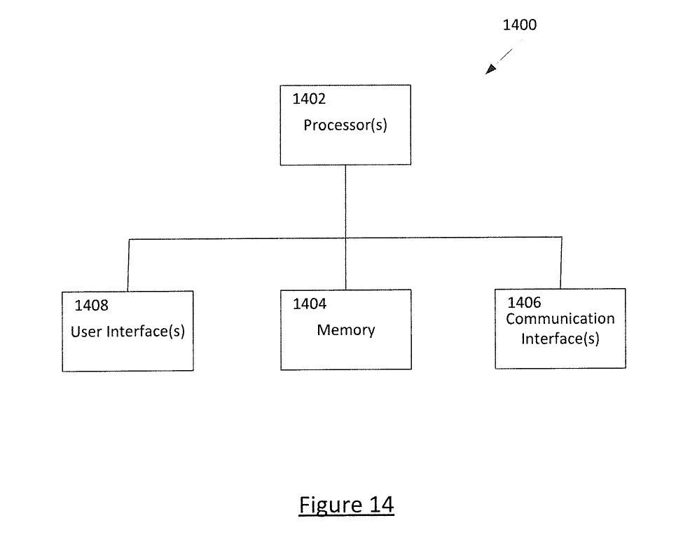

FIG. 14 is a block diagram of an apparatus that may be specifically configured in accordance with an example embodiment of the present invention.

The system of an embodiment of the present invention may include an apparatus 1400 as generally described below in conjunction with FIG. 14 for performing one or more of the operations set forth by FIGS. 1 through 13 and also described above.

It should also be noted that while FIG. 14 illustrates one example of a configuration of an apparatus 1400 for providing an electronic asthma action plan/decision support tool, numerous other configurations may also be used to implement other embodiments of the present invention. As such, in some embodiments, although devices or elements are shown as being in communication with each other, hereinafter such devices or elements should be considered to be capable of being embodied within the same device or element and thus, devices or elements shown in communication should be understood to alternatively be portions of the same device or element.

Referring now to FIG. 14, the apparatus 1400 in accordance with one example embodiment may include or otherwise be in communication with one or more of a processor 1402, a memory 1404, a communication interface 1406, and a user interface 1408.

In some embodiments, the processor (and/or co-processors or any other processing circuitry assisting or otherwise associated with the processor) may be in communication with the memory device via a bus for passing information among components of the apparatus. The memory device may include, for example, a non-transitory memory, such as one or more volatile and/or non-volatile memories. In other words, for example, the memory device may be an electronic storage device (e.g., a computer readable storage medium) comprising gates configured to store data (e.g., bits) that may be retrievable by a machine (e.g., a computing device like the processor). The memory device may be configured to store information, data, content, applications, instructions, or the like for enabling the apparatus to carry out various operations in accordance with an example embodiment of the present invention. For example, the memory device could be configured to buffer input data for processing by the processor 1402. Additionally or alternatively, the memory device could be configured to store instructions for execution by the processor.

The processor 1402 may be embodied in a number of different ways. For example, the processor may be embodied as one or more of various hardware processing means such as a coprocessor, a microprocessor, a controller, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor may include one or more processors configured in tandem via the bus to enable independent execution of instructions, pipelining and/or multithreading.

In an example embodiment, the processor 1402 may be configured to execute instructions stored in the memory device 1404 or otherwise accessible to the processor. Alternatively or additionally, the processor may be configured to execute hard coded functionality. As such, whether configured by hardware or software methods, or by a combination thereof, the processor may represent an entity (e.g., physically embodied in circuitry) capable of performing operations according to an embodiment of the present invention while configured accordingly. Thus, for example, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device configured to employ an embodiment of the present invention by further configuration of the processor by instructions for performing the algorithms and/or operations described herein. The processor may include, among other things, a clock, an arithmetic logic unit (ALU) and logic gates configured to support operation of the processor.

Meanwhile, the communication interface 1406 may be any means such as a device or circuitry embodied in either hardware or a combination of hardware and software that is configured to receive and/or transmit data from/to a network and/or any other device or module in communication with the apparatus 1400. In this regard, the communication interface may include, for example, an antenna (or multiple antennas) and supporting hardware and/or software for enabling communications with a wireless communication network. Additionally or alternatively, the communication interface may include the circuitry for interacting with the antenna(s) to cause transmission of signals via the antenna(s) or to handle receipt of signals received via the antenna(s). In some environments, the communication interface may alternatively or also support wired communication. As such, for example, the communication interface may include a communication modem and/or other hardware/software for supporting communication via cable, digital subscriber line (DSL), universal serial bus (USB) or other mechanisms.

The apparatus 1400 may include a user interface 1408 that may, in turn, be in communication with the processor 1402 to provide output to the user and, in some embodiments, to receive an indication of a user input. For example, the user interface may include a display and, in some embodiments, may also include a keyboard, a mouse, a joystick, a touch screen, touch areas, soft keys, a microphone, a speaker, or other input/output mechanisms. The processor may comprise user interface circuitry configured to control at least some functions of one or more user interface elements such as a display and, in some embodiments, a speaker, microphone and/or the like. The processor and/or user interface circuitry comprising the processor may be configured to control one or more functions of one or more user interface elements through computer program instructions (e.g., software and/or firmware) stored on a memory accessible to the processor (e.g., memory 1404, and/or the like).

As described above, FIG. 2 illustrates a flowchart of methods and systems according to an example embodiment of the invention. It will be understood that each block of the flowchart, and combinations of blocks in the flowchart, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by a memory 1404 of an apparatus employing an embodiment of the present invention and executed by a processor 1402 of the apparatus. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowchart support combinations of means for performing the specified functions and combinations of operations for performing the specified functions for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

In some embodiments, certain ones of the operations above may be modified or further amplified. Furthermore, in some embodiments, additional optional operations may be included, such as shown by the blocks with dashed outlines. Modifications, additions, or amplifications to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

That which is claimed:

1. A method comprising:
retrieving patient data;
receiving an indication of a variable to be used in the patient assessment;
determining a patient level of severity or control based at least on the patient data and the indication of the variable;
receiving a selection of a therapy step level;
determining treatment options for one or more zones based at least on the patient data, the therapy step level, and an applicable guideline;
receiving treatment selections for each of the one or more zones;
receiving a selection of condition triggers; and
generating an asthma action plan based at least in part on the patient data, the patient level of severity or control, the selected treatment options, the condition triggers, and the applicable guideline.

2. The method of claim 1 wherein the patient data comprises patient age, height, and gender.

3. The method of claim 1 wherein the indication of a variable comprises an indication of whether to use a peak flow measurement in the patient assessment,
wherein in the case where a peak flow measurement is to be used, the peak flow measurement is one of:
a calculated peak flow one, wherein the calculated peak flow is based at least in part on the patient data and one of an equation or a chart;
a prior stored best peak flow measurement for the patient; or
a received input best peak flow value.

4. The method of claim 1 wherein determining the patient level of severity or control further comprises receiving input of a symptoms assessment.

5. The method of claim 1 wherein determining the patient's level of severity or control further comprises retrieving a previous symptoms assessment and current patient medication information.

6. The method of claim 1 wherein determining one or more therapy step levels for selection comprises comparing the patient data and the patient level of severity or control to a current applicable guideline.

7. The method of claim 1 wherein determining treatment options further comprises comparing patient data and patient level of severity or control to a current applicable guideline to determine age and dose specific treatment options.

8. The method of claim 1 wherein the treatment options comprise recommended medications and dosages for each of the one or more zones.

9. The method of claim 1 wherein the treatment options further comprise an indication of formulary status for each medication.

10. The method of claim 9 wherein if the medication is on formulary, the treatment option includes an indication of insurance payment information.

11. The method of claim 1 wherein receiving treatment selections for each of the one or more zones comprises receiving one treatment option selection for a first zone and receiving one or more treatment option selections for each subsequent zone.

12. The method of claim 1 wherein the asthma action plan comprises a customized patient report.

13. The method of claim 1 wherein the asthma action plan is stored to a patient electronic medical record.

14. The method of claim 1 wherein generating the asthma action plan further comprises receiving input of additional patient-specific information to be included in the asthma action plan in a particular zone section.

15. The method of claim 1 further comprising:
generating one or more medication prescriptions based at least in part on the treatment selections; and
causing at least one of: transmission of the one or more medication prescriptions to a specified pharmacy, printing of the one or more medication prescriptions; and storing the one or more medication prescriptions to an electronic medical record.

16. An apparatus, comprising:
at least one processor; and
at least one memory including computer program instructions, the at least one memory and the computer program instructions being configured to, in cooperation with the at least one processor, cause the apparatus to at least:
retrieve patient data;
receive an indication of a variable to be used in the patient assessment;
determine a patient level of severity or control based at least on the patient data and the indication of the variable;
receive a selection of a therapy step level;
determine treatment options for one or more zones based at least on the patient data, the therapy step level, and an applicable guideline;
receive treatment selections for each of the one or more zones;
receive a selection of condition triggers; and
generate an asthma action plan based at least in part on the patient data, the patient level of severity or control, the selected treatment options, the condition triggers, and the applicable guideline.

17. The apparatus of claim 16 wherein the patient data comprises patient age, height, and gender.

18. The apparatus of claim 16 wherein the indication of a variable comprises an indication of whether to use a peak flow measurement in the patient assessment,
wherein in the case where a peak flow measurement is to be used, the peak flow measurement is one of:
a calculated peak flow one, wherein the calculated peak flow is based at least in part on the patient data and one of an equation or a chart;
a prior stored best peak flow measurement for the patient; or
a received input best peak flow value.

19. The apparatus of claim 16 wherein determining the patient level of severity or control further comprises receiving input of a symptoms assessment.

20. The apparatus of claim 16 wherein determining the patient's level of severity or control further comprises retrieving a previous symptoms assessment and current patient medication information.

21. The apparatus of claim 16 wherein determining one or more therapy step levels for selection comprises comparing the patient data and the patient level of severity or control to a current applicable guideline.

22. The apparatus of claim 16 wherein determining treatment options further comprises comparing patient data and patient level of severity or control to a current applicable guideline to determine age and dose specific treatment options.

23. The apparatus of claim 16 wherein the treatment options comprise recommended medications and dosages for each of the one or more zones.

24. The apparatus of claim 16 wherein the treatment options further comprise an indication of formulary status for each medication.

25. The apparatus of claim 24 wherein if the medication is on formulary, the treatment option includes an indication of insurance payment information.

26. The apparatus of claim 16 wherein receiving treatment selections for each of the one or more zones comprises receiving one treatment option selection for a first zone and receiving one or more treatment option selections for each subsequent zone.

27. The apparatus of claim 16 wherein the asthma action plan comprises a customized patient report.

28. The apparatus of claim 16 wherein the asthma action plan is stored to a patient electronic medical record.

29. The apparatus of claim 16 wherein generating the asthma action plan further comprises receiving input of additional patient-specific information to be included in the asthma action plan in a particular zone section.

30. The apparatus of claim 16 further comprising the at least one memory and the computer program instructions being configured to, in cooperation with the at least one processor, cause the apparatus to:
generate one or more medication prescriptions based at least in part on the treatment selections; and
cause at least one of: transmission of the one or more medication prescriptions to a specified pharmacy, printing of the one or more medication prescriptions; and storing the one or more medication prescriptions to an electronic medical record.

31. A system, comprising:
an electronic medical record system; and
a decision support tool;
wherein the decision support tool is configured at least to:
retrieve patient data from a patient electronic medical record in the electronic medical record system;
receive an indication of a variable to be used in the patient assessment;
determine a patient level of severity or control based at least on the patient data and the indication of the variable;
receive a selection of a therapy step level;
determine treatment options for one or more zones based at least on the patient data, the therapy step level, and an applicable guideline;
receive treatment selections for each of the one or more zones;
receive a selection of condition triggers; and
generate an asthma action plan based at least in part on the patient data, the patient level of severity or control, the selected treatment options, the condition triggers, and the applicable guideline.
